(19) United States
(12) Patent Application Publication
(10) Pub. No.: US 2005/0118591 A1
(43) Pub. Date: Jun. 2, 2005

(54) Diagnosis kit, dna chip, and methods for diagnosing or supervising the treatment of testicular cancer

(75) Inventors: Cengiz Tamak, Sehnde (DE); Alf-Andreas Krehan, Anzing (DE); Pia Steffens, Hannovs (DE); Stefanie Waschuetza, Hannover (DE); Veit Zieglschmid, Hannover (DE)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: ADNAGEN AG, Hannover-Langenhagen (DE)

(21) Appl. No.: 10/496,410

(22) PCT Filed: Nov. 22, 2001
(86) PCT No.: PCT/EP01/13606

(51) Int. Cl. C12Q 1/68; C12M 1/34

(52) U.S. Cl. 435/6; 435/287.2

(57) ABSTRACT

The invention relates to a diagnosis kit, a DNA chip, and to methods for diagnosing or supervising the treatment of testicular cancer, which are of greatest importance, in particular, within the scope of cancer prevention and post-treatment. The invention is essentially characterized in that the mRNA of testicular tumor markers is detected in a blood sample, whereby the tumor markers depict a tumor-associated gene expression. To this end, particularly β-hCG, AFP, PLAP or GCAP come into question. The detection of the mRNA is carried out by reverse transcription in cDNA and by subsequently amplifying selected segments of the cDNA by means of polymerase chain reaction.

The present invention concerns a diagnostic kit, a DNA chip, and a process for diagnosis or treatment control of testicular cancer. Within the framework of preliminary and later cancer care, it is of great importance to be able to detect a malignant testicular tumor or recurrent malignant testicular tumor early by means of the appearance of metastasizing tumor cells in the blood.

Testicular cancer is responsible for less than 2% of all malignant new formations of tumors in men. However, 20-30% of all cancer diseases in men under 40 years of age involve testicular cancer. The annual number of new diseases in the Federal Republic of Germany, for example, is about 3600, of which about 240 men die of testicular cancer. The highest incidence is found between age 25 and 40. Through advances in oncological therapy, long-term healing is achieved for more than 90% of all affected patients. The high survival rates in this case are based on the pronounced effectiveness of chemotherapy based of cisplatin.

In this case, it must be decided in clinical stage 1 of the disease whether the patient must be burdened with chemotherapy and/or with an operation in order to achieve a permanent successful cure. A large number of patients are treated with chemotherapy in this case, even though no certain evidence of a metastasis exists. In concepts based on pure monitoring, however, recurrence happens in 25% of cases, some with fatal outcomes.

In the research methods currently in use with cancer patients, so-called tumor markers at the protein level (immunological or enzymatic) are determined quantitatively in the blood or other body fluids of cancer patients. These detection processes are suitable for tumor diagnosis or treatment control/care only under certain conditions, however, since elevated tumor-marker values can also be caused by non-tumor diseases such as, for example, inflammations of the gastrointestinal tract, cirrhosis of the liver, virus infections, or heavy smoking.

Molecular genetic processes seem to be helpful here for detecting tumor cells in the peripheral blood, since at the start of the metastatic process, tumor cells can pass into the venous blood.

EP 0,520,794 B1 discloses a process of this kind, in which metastases can be detected in body tissue or fluids. In this case, nucleic acids are detected, for example by means of copying by a polymerase chain reaction. Now the process is based critically on the fact that the detected nucleic-acid sequence in cells of future tumor tissue is also expressed, i.e., in tumor cells and markers, under certain conditions in healthy cells of the future tissue. Another condition is that this sequence is not expressed only in cells of the tissue being studied. If, therefore, a corresponding sequence is found in the sample being studied, then this must be based on transported, i.e., metastasized cells of a tumor in another place. This process is therefore based in the end on detecting cells that should not appear in blood samples from healthy persons.

Overall, it can be stated that the currently used diagnostic methods are too imprecise when evaluation of the malignancy of residual tumors after chemotherapy has been performed in the metastasizing stages is involved. This is also true for finding evidence of hidden or residual metastases, which makes a timely assignment to one of the many primary curative therapeutic options possible.

The problem of the present invention is therefore to make available a process, a diagnostic kit, and a nucleic-acid microarray with which tumor cells can be detected in the blood in testicular-tumor diseases.

This problem is solved by the diagnostic kit according to claim 1, the microarray according to claim 20 as well as a process according to claim 22 and its application according to claim 45. Advantageous developments of the diagnostic kit, the microarray, and the process or applications will be given in the various dependent claims.

The present invention is based essentially not on detecting testicular-tumor markers at the immunological or enzymatic level in the blood of patients, but on the fact that the mRNA (messenger ribonucleic acid) of testicular-tumor markers in a blood sample is detected, whereby the tumor markers represent a gene expression associated with a tumor. As markers for testicular tumors, placenta-specific alkaline phosphatase (PLAP) and germ-cell specific alkaline phosphatase (GCAP) are used according to the invention, which are expressed by testicular tumors, epithelial glycoprotein (GA733.2 or EGP40), the high-mobility group protein isoform I-C (HMGI-C), and the gastrin-releasing peptide receptor (GRPR), are detected. The detection can be made here for only one of the markers or also for any arbitrary number of these four testicular-tumor makers in combination with one another, whereby, however, at least the mRNA of the marker GA733.2 and/or GCAP/PLAP is detected. The kit according to the invention can therefore contain oligonucleotide pairs for only one or for any arbitrary number of the four testicular-tumor markers.

Not all cases are detected by this means in which, for example because of other diseases, testicular-tumor markers are expressed in the original tissue and enter the blood circulation as proteins. Consequently, only cells are detected that, on the one hand, are themselves found in the blood sample, and on the other, express the particular testicular-tumor makers. This consequently involves tumor cells that derived from their original testicular-tumor tissue and are transported in the blood of the patients. Since the mRNA of the marker being studied is not expressed in the blood of a patient with a testicular tumor, there is a direct correlation between the appearance of the associated mRNA and a metastasis already in an early stage of the metastatic process.

Advantageously, not only the mRNA of a single testicular-tumor marker is detected, but a combination of markers is studied. In this way, it is possible, for example, to be able to detect all forms of testicular cancer through the blood because of their metastasizing cells in the blood. This means that both seminous and non-seminous forms as well as mixed tumors with portions of a seminoma, and thereby 90-95% of all malignant testicular tumors, specifically all germ-cell tumors, can be detected.

Since individual markers are expressed in different ways, depending on the therapy, it is advantageous to study a combination of tumor markers, in order to detect all tumor cells circulating in the blood. In this way, tumor cells can also be recognized when the expression of a particular marker in a patient or in a stage of the disease is relatively low, which otherwise could lead to a false negative result. The use of additional markers, however, mostly encounters limits when mononuclear blood cells have a background expression (“illegitimate transcription”) that prevents an exact analysis.

For the recognition of testicular-tumor cells, therefore, according to the invention, the following combination of markers is recommended:
GA733.2
GCAP/PLAP
HMGI-C
GRPR,
   where GA733.2 and/or GCAP/PLAP must be detected obligatorily for sure recognition of testicular-tumor cells.

In using the RT-PCR systems to detect tumor cells, specificity is a critical point because of the very high amplification rate. The slightest contaminations, such as from foreign RNA or illegitimate transcription, can make the results false in this case.

Central to the quality of the isolated RNA used as the basis for detection and the cDNA synthesized from it is enrichment of the cell fraction used for this from the blood sample used. Various methods are available for this:

a) Enrichment by Repeated Centrifugation After Erythrocyte Lysis:

1 mL EDTA-blood is lysed after addition of 5 volumes of erythrocyte-lysis buffer (“QIAmp Book Kit,” Qiagen, Hilden) for 20 min on ice. After removal of the plasma/lysate from the pelleted cells and resuspension, another centrifugation took place at 3000 G for 20 min. After the residue was removed, the pelleted leukocyte fraction is available for RNA preparation.

b) Enrichment Through Density-Gradient Centrifugation:

By means of density gradients generated through centrifugation, cells of different average volume densities can be separated from one another. Mononuclear blood cells are separated by means of a Ficoll-Hypaque gradient (Pharmacia, Uppsala, Sweden) and then washed twice with PBS/1% FCS.

c) Enrichment by Means of Antibody Labeling

Through the use of immunocytochemistry with monoclonal antibodies against tumor-cell antigens, an increase in specificity can be achieved by enriching the tumor cells with respect to the blood cells and simultaneously increasing the sensitivity of the tumor-cell detection (detection rate of 1 tumor cell in 10⁷ mononuclear blood cells). In this case, the tumor cells are separated from mononuclear blood cells by means of specific antibodies or antibody mixtures. They can be separated by means of magnetic particles (Dynal) to which the antibodies are bound. This will be described in detail in the following.

Eukaryotic cells carry a number of different molecules on their cell surface. Depending on the origin and the function of the individual cells, the combination of the expressed surface molecules differs, so that cell-type-specific patterns are formed. Antibodies are used to recognize these cell-type-specific patterns. Antibodies bind with high specificity to their antigens, to selected surface molecules in this case. This characteristic is used to recognize cells and distinguish them from one another by means of specific antibody binding, by means of their cell-type-specific patterns.

The expression of special surface proteins distinguishes tumor cells from untransformed cells of this cell type. Since this special pattern of surface antigens in tumor cells differs from the typical patterns of blood cells, tumor cells in the blood can be distinguished. In order to identify tumor cells, antibodies that specifically recognize these special surface proteins are used as tools. The specific antibody binding can be utilized for various analysis and separation methods.

Because of the intensive binding of immunoglobulins specially selected for this, it is also possible, in addition to recognizing cells by the surface epitopes, to separate the recognized cells from those that have not been recognized.

Various Separation Principles are Possible:

1. Separation Principle Based on Liquid Phase; e.g., Flow Cytometry:

For flow cytometric analysis, antibodies are coupled with fluorescent dyes. Isolated cells are directed individually in a constant liquid flow toward a light source laser). When the cells are illuminated, the fluorescent dye bound to the antibodies is stimulated and radiates light of particular wavelengths. The radiated light is detected, and the signal measured is stored digitally. The light signal can be assigned to individual cells. The antibody-labeled cells are recognized in this way and can now be separated from other cells. For the separation, the cells are isolated in the smallest drops. After antibody-labeled cells have been recognized, the corresponding drops arrive at a capturing container.

An enrichment of this kind can by made, for example, by FACS flow cytometry. In this case, for example, the mononuclear cells from the fraction enriched under b) are incubated with fluorescence-labeled mononuclear antibodies to tumor-specific surface proteins. The labeled cells are washed twice with PBS and then resuspended: 10⁷ cells in 1 mL PBS. A FACS Vantage SE flow cytometer (Becton Dickinson) is used to isolate the tumor cells. Data capture, instrument control, and data evaluation occur via the CellQuest program. The sorted cells are transferred into a 1.5-mL reaction container (filled with 1 mL PBS). The RNA can then be isolated, as will be described later.

2. Separation Principle Based on Solid Phase; e.g., Magnetic Separation:

For magnetic separation, antibodies are coupled to pseudomagnetic particles. After the pseudomagnetic particles are placed in a magnetic field, the particles wander in the magnetic field. During the movement in this magnetic field, cells to which these coupled antibodies are bound are carried along and separated from other cells.

For tumor-cell recognition by means of magnetic particles, antibodies are consequently coupled covalently to pseudomagnetic particles that have a definite number of chemically activated sites on their surface. The specificity of the separation is determined by the specificity of the antibodies. A blood sample containing tumor cells is mixed with antibody-coupled magnetic particles; then particles and blood are moved with respect to each other. The tumor cells that are recognized by the bound solid-phase antibodies and are thereby firmly attached follow the motion of the particles. In this way, it is possible, by applying a magnetic field, to pull the particles with the cells bound to them out of the blood (e.g., to the wall of the separation vessel). The blood that has been depleted of tumor cells can be replaced by other solvents, in which case the cells separated by the magnetic particles stay in place until the magnetic field is turned off/removed, and are available for further uses.

For recognizing tumor cells, specific antibody mixtures are used advantageously that are optimized either for tumor cells in general or else specifically for testicular-tumor cells. For example, a combination of antibodies MOC-31 and Ber-EP4 is suitable for recognizing tumor cells in blood.

TABLE A
Antibody Mixture 1

| Antibody | Clone | Concentration |
|---|---|---|
| Epithelial related | MOC-31 (Novocastra Co.) | 1.25 μL/10⁶ cells |
| antigen | | |
| Epithelial antigen | Ber-EP 4 (DAKO Co.) | 0.924 μg/10⁶ cells |

By means of the antibody mixture in Table A above, tumor cells are detected quite generally, but with high specificity. This is based on the selective expression of certain surface proteins that distinguish cancer cells from other cells.

Antibody mixtures of this kind show an increased sensitivity in comparison to antibodies used separately in cell recognition and cell separation, independent of the method used.

In the following, some examples of processes according to the invention, diagnostic kits used for them, and similar things will be described.

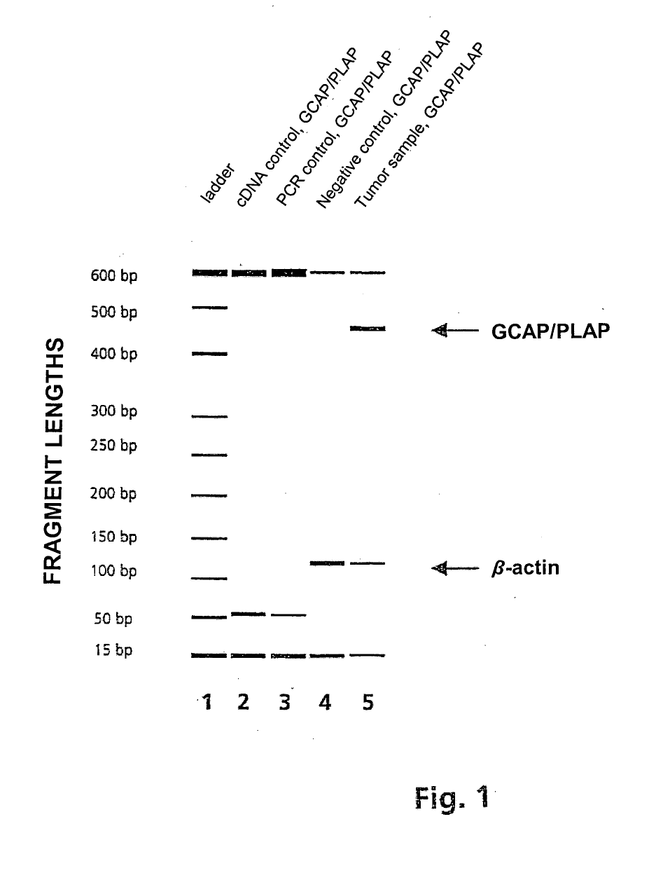

FIG. 1 shows the detection of PCR products by means of electrophoresis; and

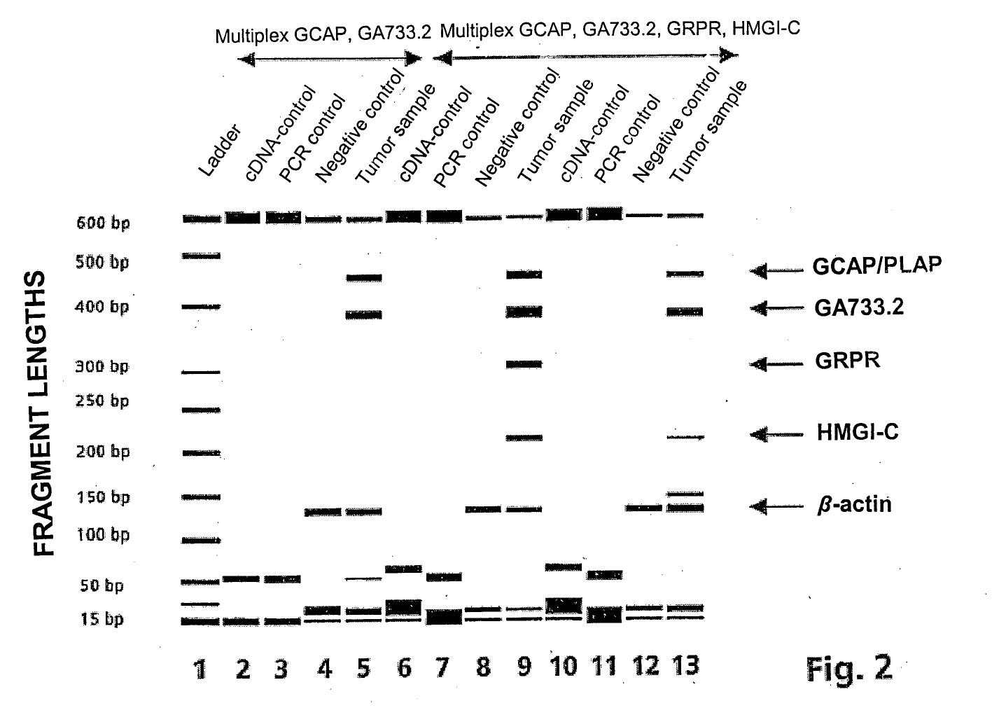

FIG. 2 shows another detection of PCR products by means of electrophoresis.

In a first step of the process according to the invention, a blood sample is taken from a patient. RNA processing is performed on one milliliter of this whole blood (as EDTA-whole blood) with THE QIAampRNA-Blood Mini Kit™ (Qiagen Company, Hilden). Contamination with genomic DNA is avoided through an additional DNA digestion with RNase-free DNase Set™ (Qiagen Company, Hilden) on the column.

Alternatively to the RNA isolation described above, it is also possible to take the isolated fraction of mononuclear blood cells, as described above, up into Trizol reagent from the company Gibco BRL, NY, USA, lyse them and homogenize them by means of a pipette. Then the aqueous phase containing the RNA is precipitated from a chloroform extraction in isopropanol at −80° C. After being washed twice in 80% ethanol, the pellet is dried in air and then resuspended in RNA-free water.

Then the RNA is denatured in a corresponding volume of water together with oligo(dT)₁₅ primer from the Promega company, Mannheim, for 5 min at 65° C., and then incubated directly on ice. A cDNA synthesis at 37° C. for one h and a subsequent inactivation of the added reverse transcriptase for 5 min at 93° C. and cooling on ice followed. All reverse transcription was performed by means of the Sensiskript™ reverse-transcriptase kit from the Qiagen Company, Hilden.

The reaction batch of 20 μL for the cDNA synthesis is given in Table 1 below:

TABLE 1
Components of the cDNA synthesis

| Components | Volumes | | Final concentration |
|---|---|---|---|
| RNA | x | μL | 5 ng/μL |
| 10X RT buffer | 2 | μL | 1X |
| dNTP mix (5 mM each) | 2 | μL | 0.5 mM each |
| Oligo(dT) primer (10 μM) | 2 | μL | 1 μM |
| Reverse transcriptase | 1 | μL | 4 U |
| RNase inhibitor | 1 | μL | 0.5 U/μL |
| RNase-free water | to 20 | μL | |

After the reverse transcription, a multiplex PCR was performed with β-actin. The corresponding reaction batch can be seen in Table 2 below.

TABLE 2
PCR batch
The PCR synthesis was performed in a 20-μL reaction batch.

| Components | Volumes | | Final concentration |
|---|---|---|---|
| cDNA | 6 | μL | |
| 10X PCR buffer* | 5 | μL | 1X |
| dNTP mix | 1 | μL | 200 mM each |
| Primer | (see | | |
| | Table 3) | | |
| Q-solution*** | 10 | μL | |
| Taq-DNA polymerase** | 0.5 | μL | 2.5 U |
| H₂O | to 50 | μL | |

*contains 15 mM MgCl₂
**HotStar Taq ™ DNA polymerase, Qiagen Co., Hilden
***Q-solution; Qiagen Co., Hilden;
(The addition of Q-solution is necessary for the detection of GCAP/PLAP.)

The PCR primers summarized in Table 3 below are used as the primer.

TABLE 3
List of PCR primers

| | |
|---|---|
| Sequence | PCR- |

| | | |
|---|---|---|
| Primer name | 5′→3′ | Product |

Tumor Marker

| | | |
|---|---|---|
| GCAP/PLAP sense | GCCACGCAGCTCATCTCCAACATG | 440 bp |
| | (SEQ ID NO: 1) | |
| GCAP/PLAP antisense | ATGATCGTCTCAGTCAGTGCCCGG | |
| | (SEQ ID NO: 2) | |
| GA733_2 sense | AATCGTCAATGCCAGTGTACTTCA | 395 bp |
| | (SEQ ID NO: 3) | |
| GA733_2 | TAACGCGTTGTGATCTCCTTCTGA | |
| antisense | (SEQ ID NO: 4) | |
| HMGI-sense | AAAGGCAGCAAAAACAAGAGTCCC | 213 bp |
| | (SEQ ID NO: 5) | |
| HMGI-antisense | CCAACTGCTGCTGAGGTAGAAATC | |
| | (SEQ ID NO: 6) | |
| GRPR sense | TCTCCCCGTGAACGATGACTGGTC | 308 bp |
| | (SEQ ID NO: 7) | |
| GRPR antisense | TGAAGACAGACACCCCAACAGAGG | |
| | (SEQ ID NO: 8) | |

Internal Control

| | | |
|---|---|---|
| β-Actin sense | CTGGAGAAGAGCTACGAGCTGCCT | 111 bp |
| | (SEQ ID NO: 9) | |
| β-Actin antisense | ACAGGACTCCATGCCCAGGAAGGA | |
| | (SEQ ID NO: 10) | |

These primers were used in the quantities and combinations listed are presented in columns in Table 4.

TABLE 4
Listing of primer quantities and primer combinations

| | Marker | | | | | |
|---|---|---|---|---|---|---|
| | | | | | | GCAP |
| | | | | | | GA733 |
| | | | GCAP | | | HMGI-C |
| Primer | GCAP | GA733_2 | GA733_2 | HMGI-C | GRPR | GRPR |
| GCAP/PLAP sense | 500 nM | | 800 nM | | | 800 nM |
| GCAP/PLAP sense | 500 nM | | 800 nM | | | 800 nM |
| GA733 2 sense | | 500 nM | 300 nM | | | 300 nM |
| GA733 2 antisense | | 500 nM | 300 nM | | | 300 nM |
| HMGI-C sense | | | | 500 nM | | 500 nM |
| HMGI-C antisense | | | | 500 nM | | 500 nM |
| GRPR sense | | | | | 500 nM | 500 nM |
| GRPR antisense | | | | | 500 nM | 500 nM |
| β-Actin-sense | 100 nM | 100 nM | 100 nM | 100 nM | 100 nM | 100 nM |
| β-Actin antisense | 100 nM | 100 nM | 100 nM | 100 nM | 100 nM | 100 nM |

The multiplex PCR was performed under the conditions given in Table 5 and at the annealing temperatures and cycles given in Table 6. In Table 5, the annealing temperature is indicated by x, where the corresponding annealing temperature from Table 6 is used.

TABLE 5
PCR conditions

| Preliminary denaturation | 95° C. 15 min |
|---|---|
| Cycle | |
| 1. Denaturation | 94° C. 1 min |
| 2. Annealing | x° C. 1 min (s.Tab.6) |
| 3. Extension | 72° C. 1 min |
| Final Extension | 72° C. 10 min |
| | 4° C. Pause |

TABLE 6
Marker-specific annealing temperatures and cycle numbers

| | Marker | | | | | |
|---|---|---|---|---|---|---|
| | | | | | | GCAP |
| | | | | | GCAP | GA733 2 |
| | GCAP | GA733 2 | HMGI-C | GRPR | GA733_2 | HMGI-C GRPR |
| Annealing- | 58° C. | 56° C. | 57° C. | 60° C. | 58° C. | 58° C. |
| Temperature | | | | | | |
| Number of cycles | 40 | 40 | 40 | 40 | 40 | 40 |

1 μL of the PCR product generated in this way was separated in an Agilent Bioanalyzer 2100 on a DNA-500 chip, and the separation result was documented by electrophoresis.

FIG. 1 shows the result of a detection by means of electrophoresis, using a DNA-500 chip (Agilent) and an Agilent Bioanalyzer 2100. Track 1 shows a 100-bp line, track 2 a cDNA-control without RNA in the batch, track 3 a PCR control without cDNA in the batch, track 4 a negative control for GCAP/PLAP with RNA from a healthy control person in the batch, and track 5 the sample from a diseased person.

The control measurement of β-actin is only positive for the two real blood samples of tracks 4 and 5, while only track 5 has the bands expected for GCAP/PLAP as a testicular-tumor marker.

Another result is shown in FIG. 2. Detection occurs in this case by means of an Agilent Bioanalyzer 2100 and a DNA-500 assay chip from Agilent. Tracks 1-6 here show a multiplex PCR of the cDNA of GCAP and GA733.2, and tracks 7-13 a multiplex PCR of cDNA of GCAP, GA733.2, GRPR, and HMGI-C. The names cDNA-control, PCR control, and negative control indicate samples without RNA, without cDNA or with RNA, respectively, from a healthy control person. It can be recognized in tracks 5, 9 or 13 that only the samples from persons suffering from testicular tumor are positive in the detection of cDNA and thereby of mRNA for GCAP/PLAP, GA733.2, GRPR, and HMGI-C.

It has therefore been shown that the corresponding testicular-tumor markers can be detected as described and claimed.

Alternatively to the methods presented here, traditional analysis methods such as agarose gel electrophoresis can also be used, naturally, in which, for example 25 μL of the PCR product described above is separated through a 2.5% agarose gel and the DNA bands are then dyed and made visible with ethidium bromide. The documentation can be carried out, for example, with the aid of the DUO store System from the Inta Co.

A fragment analysis by means of an ABI Prism 310 Genetic Analyzer (PE Applied Biosystems Co., Weiterstadt) can also be used for the evaluation. For this, a PCR with fluorescence-labeled primers is performed and then, for example, 1 μL of each PCR product is used in a 1:50 dilution.

As additional evidence, detection by means of a sequence-specific fluorescence-labeled hybridization sample is possible, which allows development of the product to be monitored after each PCR cycle. With reference to special standards, a conclusion can then be made about the quantity of starting DNA. As an alternative to block PCR, tumor detection can also be made by means of real-time PCR, using a DANN intercalating dye (e.g., LightCycler™ and CybrGreen™ from the Hoffmann-Roche Co). In addition, a quantification is made by fluorescence-based real-time PCR, for example. For this, a sequence-specific fluorescence-labeled hybridization sample was added to the PCR batch, by which, after each PCR cycle, development of the product can be monitored by means of the fluorescence emitted by it, i.e., the amplification can be followed. By reference to specific standards, conclusions can be made about the quantities of starting RNA. A quantification of the RNA associated with testicular tumor present in the blood sample and consequently a direct statement about the response to a selected method of therapy is therefore also possible. This process can be implemented, for example, with a LightCycle™ from the Roche Company, Basel, or a Taq-Man™ from the PE Applied Biosystems Company, Wieterstadt, as has been known from the literature for a long time.

Reference should also be made to the fact that the device according to the invention and the process according to the invention also make it possible to use cells that have been sorted and separated as described above further as desired. For example, they can be placed in a suitable cell-culture medium and cultured in situ.

Since the cells are intact after the separation, the characteristics of the cell membrane and the cell nucleus are preserved. This opens the possibility of performing further studies of the expression of other surface markers microscopically. For this, the sorted cells are placed on an objective carrier. Detection of additional surface markers can by done by cytochemistry or through fluorescence microscopy. Likewise, genetic analyses can be made, such as, for example, chromosome analysis by means of FISH (fluorescence in situ hybridization) or making a karyogram.

1. A diagnostic kit for diagnosis or treatment control of testicular tumors with at least two pairs of oligonucleotides (reverse primer, forward primer),
whereby the two oligonucleotides for each pair are each suitable as primers for amplification by means of a polymerase chain reaction of one of the two complementary strands of different desired DNA segments, and
whereby the one desired DNA segment is part of the cDNA for one of the tumor-marker proteins, placenta-specific alkaline phosphatase (PLAP) or germ-cell-specific alkaline phosphatase (GCAP), and the other desired DNA segment is part of the cDNA for the tumor marker protein epithelial glycoprotein 40 (GA733.2 or EGP40):

2. A diagnostic kit according to the claim 1, characterized in that it contains at least three pairs of oligonucleotides (reverse primer, forward primer),
whereby the two oligonucleotides of each pair are suitable as primers for amplification by means of a polymerase chain reaction of the two complimentary strand of the desired DNA segment, and
whereby the desired DNA segments are each part of the cDNA for various tumor-marker proteins selected from the tumor-marker proteins placenta-specific alkaline phosphatase (PLAP), germ-cell-specific alkaline phosphatase (GCAP), epithelial glycoprotein 40 (GA733.2 or EGP40), high-mobility group protein isoform I-C (HMGI-C), and/or gastrin-releasing peptide receptor (GRPR).

3. A diagnostic kit according to claim 1, characterized in that it contains at least one additional pair of oligonucleotides (reverse primer, forward primer),
whereby the two oligonucleotides of the pair are suitable as primers for amplification by means of a polymerase chain reaction of the reaction product of a polymerase chain reaction with one of the other oligonucleotide pairs.

4. A diagnostic kit according to claim 1, characterized in that it contains another pair of oligonucleotides, each of which is suitable as a primer for amplification of at least one segment of one of the two complementary strands of the cDNA for the protein β-actin as an internal control.

5. A diagnostic kit according to claim 1, characterized in that the two oligonucleotides of a pair have the following sequences, pairwise:

| | |
|---|---|
| GCCACGCAGCTCATCTCCAACATG | (SEQ ID NO: 1) |
| and | |
| ATGATCGTCTCAGTCAGTGCCCGG; | (SEQ ID NO: 2) |
| and/or | |
| AATCGTCAATGCCAGTGTACTTCA | (SEQ ID NO: 3) |
| and | |
| TAACGCGTTGTGATCTCCTTCTGA; | (SEQ ID NO: 4) |
| and/or | |
| AAAGGCAGCAAAAACAAGAGTCCC | (SEQ ID NO: 5) |
| and | |
| CCAACTGCTGCTGAGGTAGAAATC; | (SEQ ID NO: 6) |
| and/or | |
| TCTCCCCGTGAACGATGACTGGTC | (SEQ ID NO: 7) |
| and | |
| TGAAGACAGACACCCCAACAGAGG. | (SEQ ID NO: 8) |

6. A diagnostic kit according to claim 4, characterized in that at least one of each of the two oligonucleotides of a pair of oligonucleotides is labeled with fluorophores.

7. A diagnostic kit according to claim 6, characterized in that the oligonucleotides of different pairs are labeled with different fluorophores.

8. A diagnostic kit according to claim 1, characterized in that it contains a pair of oligonucleotides with the following sequences for amplification of the cDNA for β-actin:

| | |
|---|---|
| CTGGAGAAGAGCTACGAGCTGCCT | (SEQ ID NO: 9) |
| and | |
| ACAGGACTCCATGCCCAGGAAGGA. | (SEQ ID NO: 10) |

9. A diagnostic kit according to claim 8, characterized in that at least one of the two oligonucleotides of the pair is labeled with fluorophores for amplification of the cDNA for β-actin.

10. A diagnostic kit according to claim 9, characterized in that the oligonucleotide with the following sequence:

| | |
|---|---|
| CTGGAGAAGAGCTACGAGCTGCCT | (SEQ ID NO: 9) |

is labeled with the fluorophore-4,7,2″,4′,5′,7′-hexachloro-6-carboxyfluorescein.

11. A diagnostic kit according to claim 1, characterized in that it contains the substances required for performing a polymerase chain reaction.

12. A diagnostic kit according to claim 1, characterized in that it contains a buffer solution, magnesium chloride, deoxynucleotide-triphosphate, and a heat-stable polymerase as substances required for performing a polymerase chain reaction.

13. A diagnostic kit according to claim 12, characterized in that it contains a polymerase from Thermus aquaticus (Taq polymerase) as a heat-stable polymerase.

14. A diagnostic kit according to claim 1, characterized in that it contains a DNA sample with the desired DNA segment as a positive control.

15. A diagnostic kit according to claim 1, characterized in that it contains an instruction for performing the polymerase chain reaction and/or an instruction for performing a fragment analysis.

16. A diagnostic kit according to claim 1, characterized in that it contains a chart for evaluating the measurement results obtained.

17. A diagnostic kit according to claim 1, characterized in that it contains a microarray (DNA chip), whereby the array has a number of cells (fields) separate from one another and an oligonucleotide is arranged in at least one cell of the microarray that is hybridized with the desired DNA segment.

18. A diagnostic kit according to claim 17, characterized in that another oligonucleotide is arranged in at least one additional cell and the sequence of the oligonucleotide that is arranged in at least one additional cell differs from the sequence of the additional oligonucleotide.

19. A diagnostic kit according to claim 17, characterized in that an oligonucleotide is arranged in each of at least two cells, whereby the oligonucleotide arranged in various cells is hybridized with the various desired DNA segments.

20. A microarray for diagnosis or treatment control of testicular tumors with an arrangement of several cells, separated from one another, characterized in that an oligonucleotide is arranged in at least one cell that is hybridized with a DNA segment that is part of the cDNA for the tumor-marker proteins placenta-specific alkaline phosphatase (PLAP) or germ-cell-specific alkaline phosphatase (GCAP), and in at least one additional cell a nucleotide is arranged that is hybridized with a DNA section which is part of the cDNA for the tumor marker protein epithelial glycoprotein 40 (GA733.2 or EGP40).

21. A microarray according to claim 20, characterized in that various oligonucleotides are arranged in three to four cells that hybridize with each of three to four different DNA segments that are part of the cDNA of each of different tumor-marker proteins selected from placenta-specific alkaline phosphatase (PLAP), germ-cell-specific alkaline phosphatase (GCAP), epithelial glycoprotein 40 (GA733.2 or EGP40), high-mobility group protein isoform I-C (HMGI-C), and/or gastrin-releasing peptide receptor (GRPR).

22. A process for diagnosis or treatment control of testicular tumors in a human, characterized in that, in a human blood sample, the presence or absence of mRNA is detected that codes for at least the tumor-marker protein placenta-specific alkaline phosphatase (PLAP) or germ-cell-specific alkaline phosphates (GCAP), and the presence or absence is detected of mRNA that codes for the tumor marker protein epithelial glycoprotein 40 (GA733.2 or EGP40), and a conclusion is made therefrom of the presence of tumor cells in the blood sample, and therefore of a possible metastasis.

23. A process according to claim 22, characterized in that the presence or absence of at least one additional mRNA that codes for one of the tumor-marker proteins high-mobility group protein isoform I-C (HMGI-C) and/or gastrin-releasing peptide receptor (GRPR).

24. A process according claim 22, characterized in that the m-RNA is isolated directly from the whole-blood sample in a traditional manner.

25. A process according to claim 24, characterized in that a DNA digestion is performed after the isolation of the mRNA.

26. A process according claim 22, characterized in that the components of the blood sample that contain RNA are concentrated first and then this fraction is studied.

27. A process according the claim 26, characterized in that for concentrating the components containing RNA, a centrifugation of the blood sample is performed to pellet the leukocytes contained in it.

28. A process according to claim 26, characterized in that to concentrate the components containing RNA a lysis of the leukocytes contained in it is performed and then the non-lysed leukocytes are pelleted as a fraction and obtained for further diagnosis.

29. A process according to claim 26, characterized in that to concentrate the components containing RNA, at least one density-gradient centrifugation of the blood sample is used to separate and obtain the mononuclear blood cells contained in the sample.

30. A process according to claim 22, characterized in that, for the separation of testicular-tumor cells, the cells are labeled with fluorescence-labeled antibodies against tumor cells or testicular-tumor cells and separated and obtained from the sample by means of fluorescence-associated cell sorting (FACS).

31. A process according to claim 22, characterized in that, for the separation of tumor cells or testicular-tumor cells, the cells are brought into contact with magnetic particles and immobilized with antibodies on their surface aimed against tumor cells or testicular-tumor cells and the magnetic particles are then separated.

32. A process according to claim 29, characterized in that the mononuclear cells in the fraction obtained are lysed and the mRNA is separated.

33. A process according to claim 24, characterized in that the mRNA obtained is reverse-transcribed into cDNA and then the presence or absence of cDNA assigned to the tumor-marker protein is detected.

34. A process according to claim 33, characterized in that at least one predetermined segment of the cDNA is copied by a polymerase chain reaction (“PCR”) or a nested polymerase chain reaction (“nested PCR”).

35. A process according to claim 34, characterized in that, to copy the cDNA, one or more oligonucleotide pairs are used that have the following sequences:

| | |
|---|---|
| GCCACGCAGCTCATCTCCAACATG | (SEQ ID NO: 1) |
| and | |
| ATGATCGTCTCAGTCAGTGCCCGG; | (SEQ ID NO: 2) |
| and/or | |
| AATCGTCAATGCCAGTGTACTTCA | (SEQ ID NO: 3) |
| and | |
| TAACGCGTTGTGATCTCCTTCTGA; | (SEQ ID NO: 4) |
| and/or | |
| AAAGGCAGCAAAAACAAGAGTCCC | (SEQ ID NO: 5) |
| and | |
| CCAACTGCTGCTGAGGTAGAAATC; | (SEQ ID NO: 6) |
| and/or | |
| TCTCCCCGTGAACGATGACTGGTC | (SEQ ID NO: 7) |
| and | |
| TGAAGACAGACACCCCAACAGAGG. | (SEQ ID NO: 8) |

36. A process according to claim 22, characterized in that the mRNA of the protein β-actin is detected as an internal control.

37. A process according to claim 36, characterized in that the mRNA for β-actin is reverse-transcribed into cDNA and a segment of the cDNA is copied by means of a polymerase chain reaction.

38. A process according to claim 37, characterized in that, to copy the cDNA of β-actin, an oligonucleotide pair is used, whereby the oligonucleotides of the pair have the following sequences:

| | |
|---|---|
| CTGGAGAAGAGCTACGAGCTGCCT | (SEQ ID NO: 9) |
| and | |
| ACAGGACTCCATGCCCAGGAAGGA. | (SEQ ID NO: 10) |

39. A process according to claim 38, characterized in that the oligonucleotide with the sequence

| | |
|---|---|
| CTGGAGAAGAGCTACGAGCTGCCT | (SEQ ID NO: 9) |

is labeled with the fluorophore 4,7,2′,4′,5′,7′-hexachloro-6-carboxyfluorescein.

40. A process according to claim 34, characterized in that the copied cDNA segment is digested by suitable restriction enzymes and the presence or absence of the mRNA of a tumor-marker protein is determined on the basis of the cDNA fragments generated.

41. A process according to claim 34, characterized in that a gel electrophoresis of the PCR products is performed to detect the amplified cDNA segments.

42. A process according to claim 34, characterized in that a fragment analysis is performed to detect the amplified cDNA segments.

43. A process according to claim 34, characterized in that during the course of polymerase chain reaction the fluorescence generated by the products is detected and development of the product is detected (fluorescence-based real-time PCR).

44. A process according to claim 24, characterized in that a nucleotide microarray a microarray for diagnosis or treatment control of testicular tumors with an arrangement of several cells, separated from one another, characterized in that an oligonucleotide is arranged in at least one cell that is hybridized with a DNA segment that is part of the cDNA for the tumor-marker proteins placenta-specific alkaline phosphatase (PLAP) or germ-cell-specific alkaline phosphatase (GCAP), and in at least one additional cell a nucleotide is arranged that is hybridized with a DNA section which is part of the cDNA for the tumor marker protein epithelial glycoprotein 40 (GA733.2 or EGP40) is used to detect the mRNA or cDNA.

45. A process according to claim 44, characterized in that the PCR product is applied to a nucleotide microarray a microarray for diagnosis or treatment control of testicular tumors with an arrangement of several cells, separated from one another, characterized in that an oligonucleotide is arranged in at least one cell that is hybridized with a DNA segment that is part of the cDNA for the tumor-marker proteins placenta-specific alkaline phosphatase (PLAP) or germ-cell-specific alkaline phosphatase (GCAP), and in at least one additional cell a nucleotide is arranged that is hybridized with a DNA section which is part of the cDNA for the tumor marker protein epithelial glycoprotein 40 (GA733.2 or EGP40)to detect the amplified cDNA.

46. A method for diagnosis of diseases or metastasis or for treatment control of a testicular tumor comprising the diagnostic kit of claim 1.


SEQUENCE LISTING (10 sequences)

SEQ ID NO: 1 - DNA, 24 bp, Artificial
  Primer
gccacgcagctcatctccaacatg

SEQ ID NO: 2 - DNA, 24 bp, Artificial
  Primer
atgatcgtctcagtcagtgcccgg

SEQ ID NO: 3 - DNA, 24 bp, Artificial
  Primer
aatcgtcaatgccagtgtacttca

SEQ ID NO: 4 - DNA, 24 bp, Artificial
  Primer
taacgcgttgtgatctccttctga

SEQ ID NO: 5 - DNA, 24 bp, Artificial
  Primer
aaaggcagcaaaaacaagagtccc

SEQ ID NO: 6 - DNA, 24 bp, Artificial
  Primer
ccaactgctgctgaggtagaaatc

SEQ ID NO: 7 - DNA, 24 bp, Artificial
  Primer
tctccccgtgaacgatgactggtc

SEQ ID NO: 8 - DNA, 24 bp, Artificial
  Primer
tgaagacagacaccccaacagagg

SEQ ID NO: 9 - DNA, 24 bp, Artificial
  Primer
ctggagaagagctacgagctgcct

SEQ ID NO: 10 - DNA, 24 bp, Artificial
  Primer
acaggactccatgcccaggaagga